(19) United States
(12) Patent Application Publication
(10) Pub. No.: US 2022/0305123 A1
(43) Pub. Date: Sep. 29, 2022

(54) LASER ENHANCER COMPOSITION FOR IMPROVING MELASMA, WRINKLES, SKIN TIGHTENING, PORES, AND ACNE

(71) Applicant: Simfle-Stick Co., Ltd., Daegu (KR)

(72) Inventor: Kim CHANGHOON, Daegu (KR)

(73) Assignee: Simfle-Stick Co., Ltd., Daegu (KR)

(21) Appl. No.: 17/384,126

(22) Filed: Jul. 23, 2021

(30) Foreign Application Priority Data
Mar. 23, 2021 (KR) 10-2021-0037558

(51) Int. Cl. A61K 41/00; A61K 47/38; A61K 33/44; A61K 33/242; A61K 33/243; A61K 33/32; A61K 33/34; A61K 33/26; A61K 33/24; A61K 33/244; A61K 33/30

(52) U.S. Cl. CPC A61K 41/0028; A61K 47/38; A61K 33/44; A61K 33/242; A61K 33/243; A61K 33/32; A61K 33/34; A61K 33/26; A61K 33/24; A61K 33/244; A61K 33/30

(57) ABSTRACT

Proposed is a laser enhancer composition for improving melasma, wrinkles, skin tightening, pores, and acne by removing dead skin cells on an epidermal layer of the skin. The laser enhancer composition is used in a form applied to the epidermal layer during the treatment. The laser enhancer composition includes: either one or both of a peeling material and a heat conducting material that are configured not to be vaporized by a laser so that fumes are not generated; a viscous material configured to mix the peeling material 11a and the heat conducting material due to viscosity thereof; a volatile alcohol; and purified distilled water.

### CROSS REFERENCE TO RELATED APPLICATION

The present application claims priority to Korean Patent Application No. 10-2021-0037558, filed on Mar. 23, 2021, the entire contents of which is incorporated herein for all purposes by this reference.

### BACKGROUND OF THE INVENTION

### Field of the Invention

The present disclosure relates generally to a laser enhancer composition for improving melasma, wrinkles, skin tightening, pores, and acne. More particularly, the present disclosure relates to the development of an enhancer capable of: reducing or preventing the generation of fine fumes that are generated as carbon is vaporized due to high laser power during a carbon enhancer laser treatment on the epidermal layer, thereby protecting not only the health of a doctor performing the treatment, but also the health of a patient receiving the treatment; preventing the generation of the fumes in the form of fine dust, thereby reducing environmental pollution; generating no unpleasant odors such as burning odors during the operation of a laser, thereby enabling the doctor to perform the laser treatment more comfortably; and providing not only a skin peeling effect but also a heat conducting effect, thereby maximizing the effect of improving melasma, wrinkles, skin tightening, pores, and acne during the treatment.

### Description of the Related Art

In general, laser treatment for cosmetics purposes is variously called carbon laser peeling, laser soft peeling, or the like. The carbon laser peeling or laser soft peeling is a procedure that involves applying carbon, which is a laser enhancer, over the patient's face. As the laser energy is transferred into the skin using a microsecond pulse width laser (Q-switched Nd:TAG laser), the carbon heats up and vaporizes, taking with it any absorbed material including dead skin cells. The heat from the laser energy also helps improve melasma, wrinkles, skin tightening, pores, and acne.

However, when the carbon as the laser enhancer absorbs the laser energy, it is vaporized, generating fine fumes. Inhalation of these fumes may cause cancer in the human body and deteriorate lung function, and application of carbon to the skin may cause skin tumors.
Ref. 1) Morfeld P and McCunney R J, (2009). Carbon Black and lung cancer testing a novel exposure metric by multi-model inference. Am. J. Ind. Med. 52: 890-899.
Ref. 2) Harber P, Muranko H, Solis S, Torossian A, Merz B. (2003) Effect of carbon black exposure on respiratory function and symptoms.
Ref 3) J. Occup. Env. Med. 45: 144-55. ILSI Risk Science Institute Workshop: The Relevance of the Rat Lung Response to Particle to Particle Overload for Human Risk Assessment.
Ref 4) Inh. Toxicol. 12:1-17 (2000). International Agency for Research on Cancer: IARC Monographs on the Evaluation of Carcinogenic Risks to Humans (2010), Vol. 93, February 1-14, 2006, Carbon Black, Titanium Dioxide, and Talc. Lyon.

In order to prevent inhalation of such fumes, conventionally, a doctor performing the treatment removes carbon with a laser held in one hand while suctioning generated fumes with a separate suction instrument held in the other hand, or a nurse has to assist in handling the suction instrument. However, these fumes may not be completely removed, causing health problems to the doctor, the nurse, and a patient as well as potential environmental pollution.

For example, according to Korean Patent No. 10-1446706, there is disclosed “Cosmetic composition for skin rejuvenation”. The composition contains fibroblast growth factor-9 as an active ingredient for pre-treatment using a carbon enhancer and application of the carbon enhancer to the skin after laser treatment, and the laser treatment is performed at a wavelength of 10,300 to 10,900 nm.

Since the composition is used to rejuvenate the skin through the laser treatment, when the skin is laser-treated with the carbon applied thereon, it is necessary to completely remove the carbon and dead skin cells through a separate suction instrument due to the generation of fine fumes. However, a complete removal of the carbon and dead skin cells may not be accomplished, which may not guarantee the safety of the respiratory system for those involved in the treatment, including a doctor, a patient, a nurse, etc.

The foregoing is intended merely to aid in the understanding of the background of the present disclosure, and is not intended to mean that the present disclosure falls within the purview of the related art that is already known to those skilled in the art.

### DOCUMENTS OF RELATED ART

(Patent document 1) Korean Patent No. 10-1446706 “Cosmetic Composition for Skin Regeneration”

### SUMMARY OF THE INVENTION

Accordingly, the present disclosure has been made keeping in mind the above problems occurring in the related art, and an objective of the present disclosure is to provide a laser enhancer capable of: reducing or preventing the generation of fine fumes that are generated as carbon is vaporized due to high laser power during a carbon enhancer laser treatment on the epidermal layer, thereby protecting not only the health of a doctor performing the treatment, but also the health of a patient receiving the treatment; preventing the generation of the fumes in the form of fine dust, thereby reducing environmental pollution; generating no unpleasant odors such as burning odors during the operation of a laser, thereby enabling the doctor to perform the laser treatment more comfortably; and providing not only a peeling effect but also a heat conducting effect, thereby maximizing the effects of improving melasma, wrinkles, skin tightening, pores, and acne during the treatment.

In order to achieve the above objective, according to one aspect of the present disclosure, there is provided a laser enhancer composition for removing dead skin cells on the epidermal layer during a laser peeling treatment, thereby improving melasma, wrinkles, skin tightening, pores, and acne, the laser enhancer composition being used in a form applied to the epidermal layer during the treatment. The laser enhancer composition may include: either one or both of a peeling material and a heat conducting material that are configured not to be vaporized by a laser so that fumes are not generated; a viscous material configured to mix the peeling material and the heat conducting material due to viscosity thereof; a volatile alcohol; and purified distilled water.

The peeling material may include diamond in a powder form with a predetermined particle size so as to enable skin peeling, and the heat conducting material may include any one of a magnetic substance, gold, and platinum that are in a powder form with a predetermined particle size or include a mixture thereof so as to enable heat conduction.

Each of the peeling material and the heat conducting material may have a particle size of 1 to 5 μm to maximize a peeling effect and to obtain an appropriate amount of absorption.

The magnetic substance of the heat conducting material may include: a metal material selected from the group consisting of Pt, Pd, Ag, Cu, and Au or a mixture of metals selected from the same group; and a magnetic material selected from the group consisting of Co, Mn, Fe, Ni, Gd, Mo, MM′₂O₄, and MₓO_{y} (M and M′ each independently represents Co, Fe, Ni, Mn, Zn, Gd, or Cr, 0<x≤3, and 0<y≤5), or a magnetic alloy selected from the group consisting of CoCu, CoPt, FePt, CoSm, NiFe, and NiFeCo.

The viscous material may include carboxymethyl cellulose (CMC).

The laser enhancer composition may include: 1 to 3 wt % of either one or both of the peeling material and the heat conducting material; 1 to 3 wt % of the viscous material; 43 to wt % of the alcohol; and 45 to 55 wt % of the distilled water.

Prior to the description, it will be understood that all terms including technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein. Accordingly, embodiments disclosed in this specification and drawings are only exemplary and do not cover all the technical spirits disclosed in this specification, and thus it is to be understood that various modifications, equivalents, and substitutions may be made in the embodiments at the time when this application is filed.

As is apparent from the configuration and operation described above, the present disclosure provides the following effects. First, by reducing or preventing the generation of fine fumes that are generated as carbon is vaporized due to high laser power during a carbon enhancer laser treatment on the epidermal layer, it is possible to protect not only the health of a doctor performing the treatment, but also the health of a patient receiving the treatment. Second, by preventing the generation of the fumes in the form of fine dust, it is possible to reduce environmental pollution. Third, unpleasant odors such as burning odors are not generated during the operation of a laser, so it is possible to enable the doctor to perform the laser treatment more comfortably. Fourth, by providing not only a peeling effect but also a heat conducting effect, it is possible to maximize the effects of improving melasma, wrinkles, skin tightening, pores, and acne during the treatment.

### BRIEF DESCRIPTION OF THE DRAWINGS

The above and other objectives, features, and other advantages of the present disclosure will be more clearly understood from the following detailed description when taken in conjunction with the accompanying drawings, in which:

FIGS. 1A to 1D are cross-sectional views sequentially illustrating the process of laser treatment using a laser enhancer composition for improving melasma, wrinkles, skin tightening, pores, and acne according to the present disclosure.

FIG. 2 shows a laser irradiation of gold of heat conducting material.

FIG. 3 shows a heat transfer test on gold cage particles and carbon particles.

FIG. 4 shows an absorption test by particle size to determine preferred particle sizes that allow peeling material and heat conducting material of laser enhancer composition to be absorbed only in epidermal layer.

FIG. 5 shows a test showing that gold cage particles of laser enhancer composition form group by laser to have increased thermal conductivity.

FIG. 6 shows carbon before and after treatment.

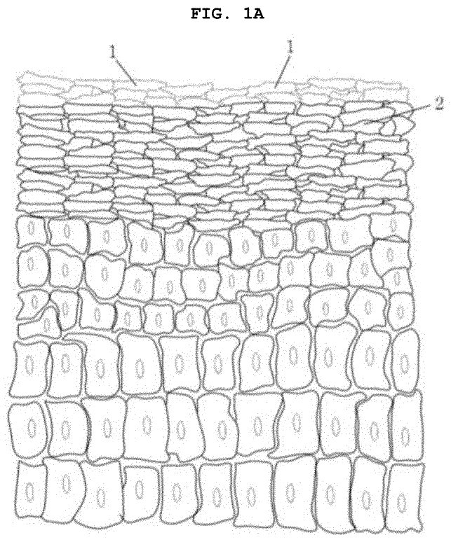

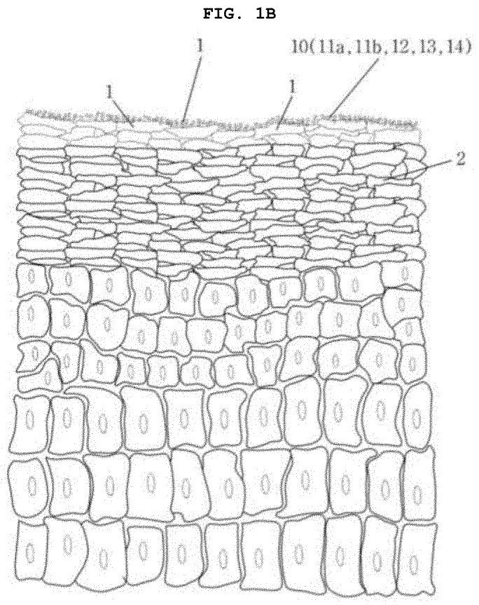

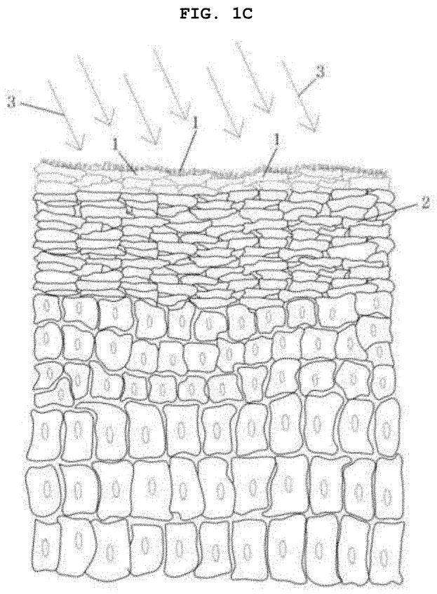

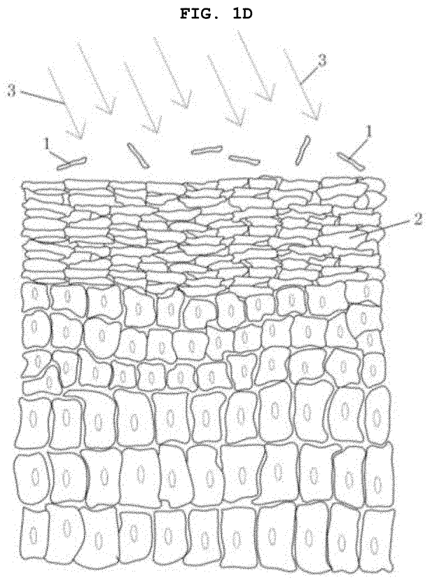

### DETAILED DESCRIPTION OF THE INVENTION

Hereinafter, exemplary embodiments of the present disclosure will be described in detail with reference to the accompanying drawings.

The present disclosure relates to a laser enhancer composition for use in removing dead skin cells 1 on an epidermal layer 2 of the skin during a laser peeling treatment. The laser enhancer composition 10 is used in a form applied to the epidermal layer 2 during the treatment and includes a peeling material 11a, a heat conducting material 11b, a viscous material 12, a volatile alcohol 13, and purified distilled water 14.

The peeling material 11a according to the present disclosure is configured to enable skin peeling and the heat conducting material 11b is configured to enable heat conduction. The peeling material 11a and the heat conducting material 11b constituting the laser enhancer composition 10 are components for removing the dead skin cells 1 on the epidermal layer 2, and have the ability not to be vaporized by a laser 3 so that fumes are not generated. The peeling material 11a and the heat conducting material 11b of the laser enhancer composition 10 perform their respective roles. The peeling material 11a performs a peeling action to exfoliate the patient's skin when a doctor performs a peeling operation through the laser 3, and the heat conducting material 11b performs a heat conducting action to transfer heat to the epidermal layer 2 when the doctor performs a heating operation through the laser 3. The peeling material 11a and the heat conducting material 11b may be selectively used depending on the condition of the patient's skin, and may be used in conjunction with each other, which will be described later. Conventionally, in the case of laser treatment using carbon, the heat generated from a laser causes the carbon to vaporize, generating fine fumes in the air. These fumes may cause various respiratory diseases such as cancer when inhaled into the respiratory system of the doctor, nurse, and patient. However, the peeling material 11a and the heat conducting material 11b according to the present disclosure are not vaporized by the heat from the laser 3 during the treatment, so it is possible to prevent the generation of fine fumes while enabling a safe laser treatment.

In this case, the peeling material 11a of the laser enhancer composition 10 includes diamond in a powder form with a predetermined size so as to enable peeling. The heat conducting material 11b includes any one of a magnetic substance, gold, and platinum that are in a powder form with a predetermined size or a mixture of at least one thereof so as to enable heat conduction. As described above, the peeling material 11a and the heat conducting material 11b of the laser enhancer composition 10 perform their respective roles. The peeling material 11a performs the peeling action to exfoliate the patient's skin when the doctor performs the peeling operation through the laser 3, and the heat conducting material 11b performs the heat conducting action to transfer heat to the epidermal layer 2 to stimulate collagen growth in the tissue when the doctor performs the heating operation through the laser 3. Therefore, the peeling material 11a includes diamond to enable peeling, and the heat conducting material 11b includes the magnetic substance, gold, or platinum to enable heat conduction. The peeling material 11a and the heat conducting material 11b are not vaporized by the laser 3. Any one of the peeling material 11a and the heat conducting material 11b may be selected and used, or the peeling material 11a and the heat conducting material 11b may be used in conjunction with each other. The heat conducting material 11b may include any one of the magnetic substance and gold, or a mixture of at least one thereof. Therefore, it is possible to perform the peeling action and the heat conducting action selectively or simultaneously without the problem of fume generation. The magnetic substance may be made of a metal material, which will be described later. In addition, diamond, gold, and platinum are excellent in heat conduction, and efficiently remove the dead skin cells 1 without being melted or vaporized by the laser 3, thereby enabling a safe laser treatment.

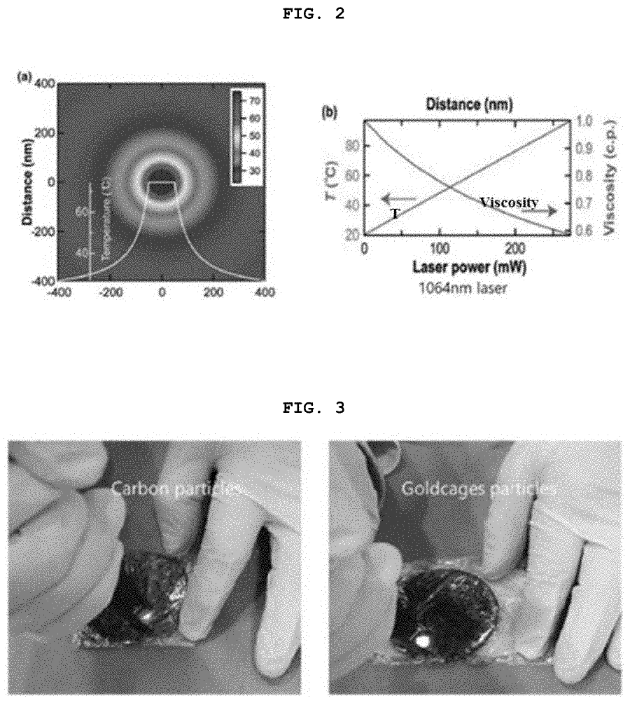

FIG. 2 illustrates an example. When gold cage particles of the heat conducting material 11b were irradiated with a Q-switched Nd:TAG laser 3 at a fluence of 1.6 J/cm² and a pulse duration of 5 ns, the gold cage particles were heated to approximately 70° C. This result indicates that it is possible to maximize the effects of improving skin whitening, melasma, wrinkles, skin tightening, pores, acne, and skin peeling.

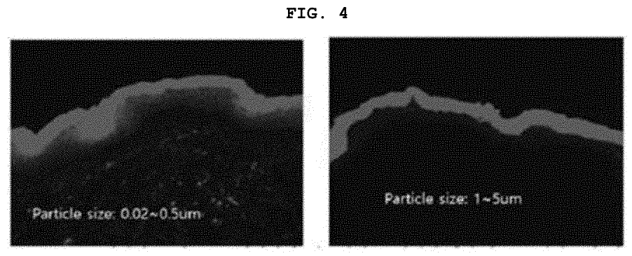

In FIG. 3, as a result of applying the laser 3 used during the treatment to the gold cage particles of the heat conducting material 11b and applying the same laser 3 to carbon particles, there was three times or more difference in thermal conductivity between the gold cage particles and the carbon particles.

In addition, an absorption test by particle size to determine preferred particle sizes that allows peeling material and heat conducting material of laser enhancer composition to be absorbed only in epidermal layer was conducted, and the results are shown in FIG. 4.

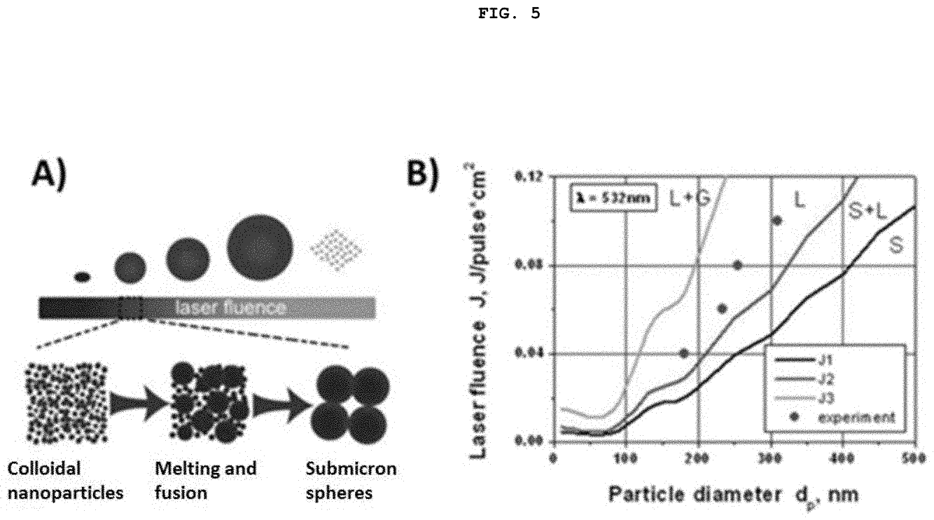

Furthermore, a test showing that gold cage particles of laser enhancer composition form group by laser to have increased thermal conductivity was conducted, and the result is shown in FIG. 5.

In this case, each of the peeling material 11a and the heat conducting material 11b has a particle size of 1 to 5 μm to maximize a peeling effect and obtain an appropriate amount of absorption. With the particle size of 1 to 5 μm, it is possible to not only realize a maximum peeling effect and an appropriate amount of absorption, but also enable the peeling material 11a and the heat conducting material 11b to be efficiently absorbed between the dead skin cells 1 on the epidermal layer 2. When the particle size exceeds 5 μm, the effect of the peeling action is remarkably deteriorated. On the other hand, when the particle size is equal to or less than 1 μm, a large amount of particles is absorbed into the skin and thus tends to enter the respiratory system, etc. thus causing side effects. Therefore, such problems can be solved with the particle size in the above range. Within the range of 1 to 5 μm, the particle size may be variously set to an appropriate level depending on the skin condition or the skin area of the patient.

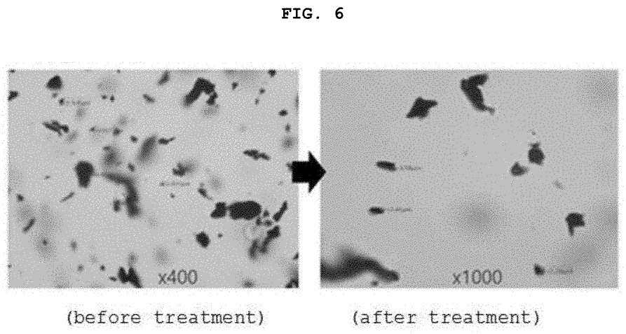

Conventionally, carbon has a size of 5 to 40 μm before the treatment. During the treatment, the carbon is vaporized by the laser, generating fumes having a size of equal to or less than 1 μm, which is comparable to that of ultrafine dust. After the treatment, the resulting carbon fumes remain in the air. See FIG. 6. The fumes are highly toxic because they may cause respiratory diseases such as cancer when inhaled into the body through the respiratory system. The fumes refer to those floating in the air in the form of fine solid particles generated by solidification of vapors of solid substances or chemical reactions of gaseous substances. The fumes generally have a size equal to or less than 1 μm in diameter, which is smaller than that of dust, and thus are difficult to settle to the ground and easily enter the respiratory system.

In addition, the magnetic substance of the heat conducting material 11b includes: a metal material of any one selected from the group consisting of Pt, Pd, Ag, Cu, and Au or of a mixture of at least one thereof; a magnetic material selected from the group consisting of Co, Mn, Fe, Ni, Gd, Mo, MM′₂O₄, and MₓO_{y} (M and M′ each independently represents Co, Fe, Ni, Mn, Zn, Gd, or Cr, 0<x≤3, 0<y≤5); or a magnetic alloy selected from the group consisting of CoCu, CoPt, FePt, CoSm, NiFe, and NiFeCo. The magnetic substance is magnetic nanoparticles or magnetic nanoclusters, which refer to a group of individual nanoparticles, and may exist in a dispersed form or a core form in dielectric particles. The magnetic nanoparticles are preferably a metal material having magnetic properties, a magnetic material, or a magnetic alloy. It is preferable that the metal material is one selected from the group consisting of Pt, Pd, Ag, Cu, and Au or a mixture of at least one thereof, the magnetic material is one selected from the group consisting of Co, Mn, Fe, Ni, Gd, Mo, MM′₂O₄, and MₓO_{y} (M and M′ each independently represents Co, Fe, Ni, Mn, Zn, Gd, or Cr, 0<x≤3, 0<y≤5) or a mixture of at least one thereof, and the magnetic alloy is one selected from the group consisting of CoCu, CoPt, FePt, CoSm, NiFe, and NiFeCo or a mixture of at least one thereof.

In addition, the viscous material 12 according to the present disclosure is configured to mix the heat conducting material 11b due to viscosity thereof. The viscous material 12 has a viscosity sufficient to mix the heat conducting material 11b in the form of powder with a predetermined size, and enable adhesion of the heat conducting material 11b to the patient's skin.

In this case, the viscous material 12 includes carboxymethyl cellulose (CMC). The viscous material 12 determines the overall viscosity of the laser enhancer composition 10 through carboxymethyl cellulose (CMC) as a water-soluble thickener, and also prevents penetration of the laser 3 into the patient's skin, i.e., the epidermal layer 2. The carboxymethyl cellulose (CMC) of the viscous material 12 serves to mix the overall components of the laser enhancer composition 10 with each other.

In addition, the volatile alcohol 13 is used in the present disclosure. When the laser enhancer composition 10 is applied to the epidermal layer 2 of the patient's skin, the alcohol 13 volatilizes over a predetermined period of time. In this case, the alcohol 13 volatilizes, taking with it excessive sebum in the epidermal layer 2, without causing any skin troubles since the alcohol 13 does not contain components such as oil causing skin troubles.

In addition, the purified distilled water 14 is used in the present disclosure. When normal water is used, various substances such as calcium, magnesium, copper, and iron contained therein may cause a deterioration in action. This problem can be solved with the use of the purified distilled water 14 in the present disclosure.

The laser enhancer composition 10 includes 1 to 3 wt % of either one or both of the peeling material 11a and the heat conducting material 11b, 1 to 3 wt % of the viscous material 12, 43 to 50 wt % of the alcohol 13, and 45 to 55 wt % of the distilled water 14. It is preferable that the laser enhancer composition 10 includes 1 wt % of either one or both of the peeling material 11a and the heat conducting material 11b, 1 wt % of the viscous material 12, 48 wt % of the alcohol 13, and 50 wt % of the distilled water 14. However, since each patient has a different skin type, the components of the laser enhancer composition 10 may be mixed at various mixing ratios depending on situations. The mixing ratios may be set depending on the amount of the dead skin cells 1 on the epidermal layer 2, and may be set depending on the amount of the dead skin cells 1 to be removed.

As described above, the present disclosure relates to the laser enhancer composition for treating age spots, wrinkles, acne scars, and pimples and for tightening pores and skin. The present disclosure provides the following effects. First, by reducing or preventing the generation of fine fumes that are generated as carbon is vaporized due to high laser power during a carbon enhancer laser treatment on the epidermal layer, it is possible to protect not only the health of the doctor performing the treatment, but also the health of the patient receiving the treatment. Second, by preventing the generation of the fumes in the form of fine dust, it is possible to reduce environmental pollution. Third, no unpleasant odors such as burning odors are generated during the operation of the laser, so it is possible to enable the doctor to perform the laser treatment more comfortably. Fourth, by providing not only a peeling effect but also a heat conducting effect, it is possible to maximize the effects of improving melasma, wrinkles, skin tightening, pores, and acne during the treatment.

The present disclosure, however, is not limited to only the embodiments set forth herein, and it is apparent to those skilled in the art that various changes and modifications may be made therein without departing from the spirit and scope of the present disclosure. Therefore, such changes and modifications may be included within the scope of the present disclosure as defined by the appended claims.

What is claimed is:

1. A laser enhancer composition for removing dead skin cells on an epidermal layer during a laser peeling treatment, thereby improving melasma, wrinkles, skin tightening, pores, and acne, the laser enhancer composition being used in a form applied to the epidermal layer during the treatment, the laser enhancer composition comprising:
either one or both of a peeling material and a heat conducting material that are configured not to be vaporized by a laser so that fumes are not generated;
a viscous material configured to mix the peeling material and the heat conducting material due to viscosity thereof;
a volatile alcohol; and
purified distilled water.

2. The laser enhancer composition of claim 1, wherein the peeling material comprises diamond in a powder form with a predetermined particle size so as to enable skin peeling, and
the heat conducting material comprises any one of a magnetic substance, gold, and platinum that are in a powder form with a predetermined particle size or comprises a mixture thereof so as to enable heat conduction.

3. The laser enhancer composition of claim 2, wherein each of the peeling material and the heat conducting material has a particle size of 1 to 5 μm to maximize a peeling effect and to obtain an appropriate amount of absorption.

4. The laser enhancer composition of claim 2, wherein the magnetic substance of the heat conducting material comprises:
a metal material selected from the group consisting of Pt, Pd, Ag, Cu, and Au or a mixture of metals selected from the same group; and
a magnetic material selected from the group consisting of Co, Mn, Fe, Ni, Gd, Mo, MM′₂O₄, and MₓO_{y} (M and M′ each independently represents Co, Fe, Ni, Mn, Zn, Gd, or Cr, 0<x≤3, and 0<y≤5), or a magnetic alloy selected from the group consisting of CoCu, CoPt, FePt, CoSm, NiFe, and NiFeCo.

5. The laser enhancer composition of claim 1, wherein the viscous material comprises carboxymethyl cellulose (CMC).

6. The laser enhancer composition of claim 1, wherein the laser enhancer composition comprises:
1 to 3 wt % of either one or both of the peeling material and the heat conducting material;
1 to 3 wt % of the viscous material;
43 to 50 wt % of the alcohol; and
45 to 55 wt % of the distilled water.
